(19) United States
(12) Patent Application Publication
(10) Pub. No.: US 2007/0129732 A1
(43) Pub. Date: Jun. 7, 2007

(54) Spring-Mass Surgical System

(76) Inventor: Jaime Zacharias, Vitacura (CL)

Correspondence Address: LAW OFFICE OF JAY R. YABLON, 910 NORTHUMBERLAND DRIVE, SCHENECTADY, NY, 12309-2814, US

(21) Appl. No.: 11/164,507

(22) Filed: Nov. 28, 2005

(51) Int. Cl. A61F 9/00

(52) U.S. Cl. 606/107

(57) ABSTRACT

A high-speed surgical handpiece (10) of the kind suitable for vitreoretinal surgery having a cutter (44) and an actuator (310). The cutter (44) is a guillotine-type cutter activated by a spring-mass system excited at harmonic frequency by a piezoelectric actuator (310) that receives a driving signal from a driving controller. The controller can have control and display units with a plurality of input mechanisms receiving input from a user. The control unit produces a piezoelectric actuator output signal to excite the spring-mass system at harmonic frequency. Fast cutting rates with reduced duty cycle as well as a proportional mode of operation are available. Low degrees of vibration and noise generation are produced.

### FIELD OF THE INVENTION

This invention is related to electrically operated surgical systems, and more particularly to a surgical system of the kind suitable for vitreoretinal surgery powered by a resonating piezoelectric mechanism.

### BACKGROUND OF THE INVENTION

The intraocular portion of current vitrectomy probes typically consists in a closed end outer tube having a distal end sideport to aspirate the vitreous, and an inner tube that oscillates axially during operation in a way that the distal end sharp edge can displace with a cutting action across said sideport.

Oscillation of the inner tube is typically provided by pneumatic turbines and electric rotary motors. Also, diaphragm based pneumatic systems have been used operated by fast changes in pressure levels inside a gas chamber at the handpiece proximal portion. These changes in pressure levels are console driven typically consisting in the alternation of positive and negative pressure cycles at the operation frequency desired for the cutter.

Vacuum applied by a vacuum source in fluid communication with the hollow oscillating tube aspirates the vitreous into the sideport and the axially oscillating inner tube distal end sharp edges cut the vitreous allowing the aspiration and removal of the vitreous and any other intraocular material to be removed. A fluid source in direct communication with the intraocular cavity can provide pressurized balanced salt solution to replace the volume of the removed vitreous.

There would be advantage in increasing the speed of operation of vitrectomy cutters as less traction would be applied to the vitreous body and the displacement of tissue into the aspirating sideport would be more controlled and continuous. Currently available pneumatic vitreous cutters can operate up to 2.500 cuts per minute but typically exhibit a reduced duty cycle.

Electrically driven vitreous cutters can operate at higher speeds, up to 3.000 cuts per minute, but are typically heavy, delicate and vibrate during operation. These details have been exposed in U.S. Pat. No. 6,575,990 the one I incorporate here as a reference. U.S. Pat. No. 6,875,221 and USPTO co-pending application Ser. No. 11/164,164 are also cited here with its accompanying references to provide background for the present description.

Typically, the speed of the cutting blade of currently available electrically operated vitrectomy handpieces is proportional to the cut rate. When operating at low cut rates, the blade traverses the cutting sideport at a lower speed than when operating at higher cutting rates. This mode of operation is related to the rotary coupled mechanism of many electric vitrectomy handpieces.

Pneumatic handpieces exhibit a progressive increase of the closed-to-open ratio as the cut rate is increased, as physical limitations apply to recycle the guillotine cutter with its biasing preloading spring. One limitation of pneumatic vitreous cutters operating at high speed is that the closed-to-open ratio progressively increases as the operating speed is increased.

This increase of the portion of the cycle where the sideport is closed with respect to the duration of one full cycle reduces cutter efficiency as less time is available for vacuum to aspirate vitreous tissue into the sideport for the cutting and aspirating action.

The reduced efficiency increases surgical time increasing complications such as post-vitrectomy cataract formation and reduces operating room turn around.

Another limitation of current vitrectomy cutters operating at high speed is that there can be vibration of the tip of the surgical instrument related to movements of the internal mechanisms used to power the cutting edges.

Another limitation of current vitrectomy cutters is that regulation of the open sideport area cannot be adjusted or requires manual mechanical adjustments at handpiece level.

Still another limitation of current vitrectomy cutters operated at high speed is that the vibration of the internal mechanisms used to power the cutting edges produces noise.

There is still a need for vitrectomy cutters that can operate in the high speed range to cut the vitreous.

Also, there is a need for vitrectomy cutters providing maximum sideport open ratios preferably above 50% when operating at cut rates above 1.500 cuts per minute.

Also, there is a need for electric vitrectomy cutters that allow an operator to adjust the maximally open sideport dimensions.

Also there is the need for a high speed vitreous cutting handpiece that is lightweight, operates silently and produces a minimum of vibration.

Also there is the need for a high speed vitreous cutting handpiece that is mechanically simple allowing repeated sterilization and providing reduced wear and failure rates.

It is an object of the present invention to provide a vitreous cutter mechanism that allows a fast cutting speed of the cutting edge across the aspirating sideport.

It is another object of the present invention to provide a vitrectomy probe that can operate efficiently at speeds above 2.000 cuts per minute.

It is still another object of the present invention to provide a vitreous cutter handpiece where the open sideport ratio is above 50% at high operating frequencies.

It is still another object of the present invention to provide an electric vitreous cutter handpiece that allows adjustment of the position of the cutting border within a vitrectomy handpiece cutting sideport to regulate the effective area of the open sideport.

It is still another object of the present invention to provide a vitreous cutter handpiece that also allows an operator to displace the cutting border across a vitrectomy sideport following a footpedal command or other proportional user interface inputs.

It is still another object of the present invention to provide a vitreous cutter handpiece that operates silently and that produces a minimum of actuator-related vibration during operation.

It is still another object of the present invention to provide a vitreous cutter handpiece that is lightweight and resistant to sterilization.

It is still another object of the present invention to provide a vitreous cutter mechanism that is mechanically simple with reduced wear and failure rates.

### BRIEF DESCRIPTION OF THE DRAWINGS

The features of the invention believed to be novel are set forth in the appended claims. The invention, however, together with further objects and advantages thereof, may best be understood by reference to the following description taken in conjunction with the accompanying drawing(s) summarized below.

FIG. 1 depicts a schematic view of a vitrectomy system incorporating the handpiece of the present invention.

FIG. 2 depicts a schematic external view of the vitrectomy handpiece.

FIG. 3A is a schematic lateral view of the handpiece of the present invention with a direct piezoelectric actuator and attached spring-mass system in compressed state driving the guillotine to the open position.

FIG. 3B is a schematic lateral view of the handpiece of the present invention with the direct piezoelectric actuator and attached spring-mass system in expanded state driving the guillotine to the closed position.

FIG. 4A is a schematic lateral view of the handpiece of the present invention with an amplified piezoelectric actuator and spring-mass system in compressed state driving the guillotine to the open position.

FIG. 4B is a schematic lateral view of the handpiece of the present invention with an amplified piezoelectric actuator and spring-mass system in expanded state driving the guillotine to the closed position.

FIG. 5 is a schematic lateral view of the handpiece of the present invention with a direct piezoelectric actuator and attached spring-mass system mounted on an operator adjustable screw based support to regulate the maximally open sideport dimensions.

FIG. 6 is a schematic lateral view of the handpiece of the present invention with a direct piezoelectric actuator and attached spring-mass system mounted on an axially adjustable support operated by a linear actuator to regulate the maximally open sideport dimensions.

FIG. 7 is a schematic lateral view of the handpiece of the present invention including a twin mass system to provide axial vibration canceling.

FIG. 8 is a schematic diagram of a vitrectomy system incorporating the handpiece of the present invention.

FIG. 9 includes a graph depicting the typical behavior of an un-damped spring-mass system of the present invention excited at harmonic frequency.

### LIST OF REFERENCE NUMERALS

Surgical handpiece 10, vitrectomy probe proximal end 11, vitrectomy probe 12, vitrectomy probe distal end 13, vitrectomy probe sideport 14, guillotine cutting edge 15, surgical handpiece body 16, detachable head 17, aspiration port 18, aspiration tubing 19, surgical handpiece cable 20, actuator driver cable 21, piezoelectric actuator cable 22, position sensor cable 23, Vibration sensor cable 24, body-head coupling 29, amplified piezoelectric actuator 30, actuator connection pad 32, amplified piezoelectric actuator leveraging fame 34, piezoelectric actuator 36, interlock coupling 40, aspiration duct 42, guillotine 44, surgical system console 70, user interface 71, controls 72, display 73, footpedal 74, footpedal cable 75, footpedal connector 76, aspiration tubing connector 77, surgical handpiece cable connector 78, position sensor 80, position sensor cable 81, pressurized balanced salt solution 90, solenoid 92, infusion tubing 94, eye 96, irrigation incision 97, vitrectomy probe incision 98, spring 300, rod guide 302, cavity guide 304, mass 306, coupling connector 306, piezoelectric actuator 310, spring 400, rod guide 402, cavity guide 404, mass 406, piezoelectric actuator 408, fixating assembly 450, bias adjustment screw 500, piezoelectric actuator support 502, thread 504, linear actuator 540, stopper/damper 600, spring 800, male guide 802, female guide 804, mass 806.

### SUMMARY OF THE INVENTION

A vitrectomy handpiece powered by a piezoelectric actuator driving a guillotine based vitreous cutter using a spring-mass mechanism under harmonic excitation to increase stroke and provide high speed of operation.

### DETAILED DESCRIPTION

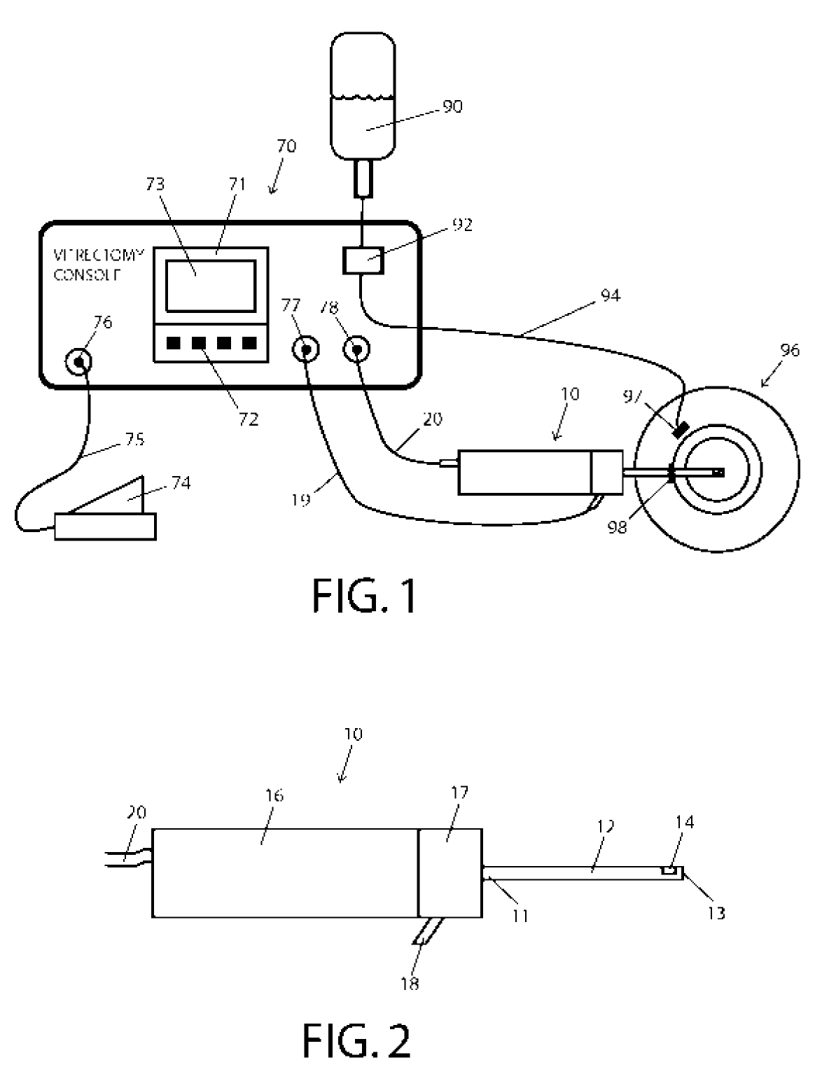

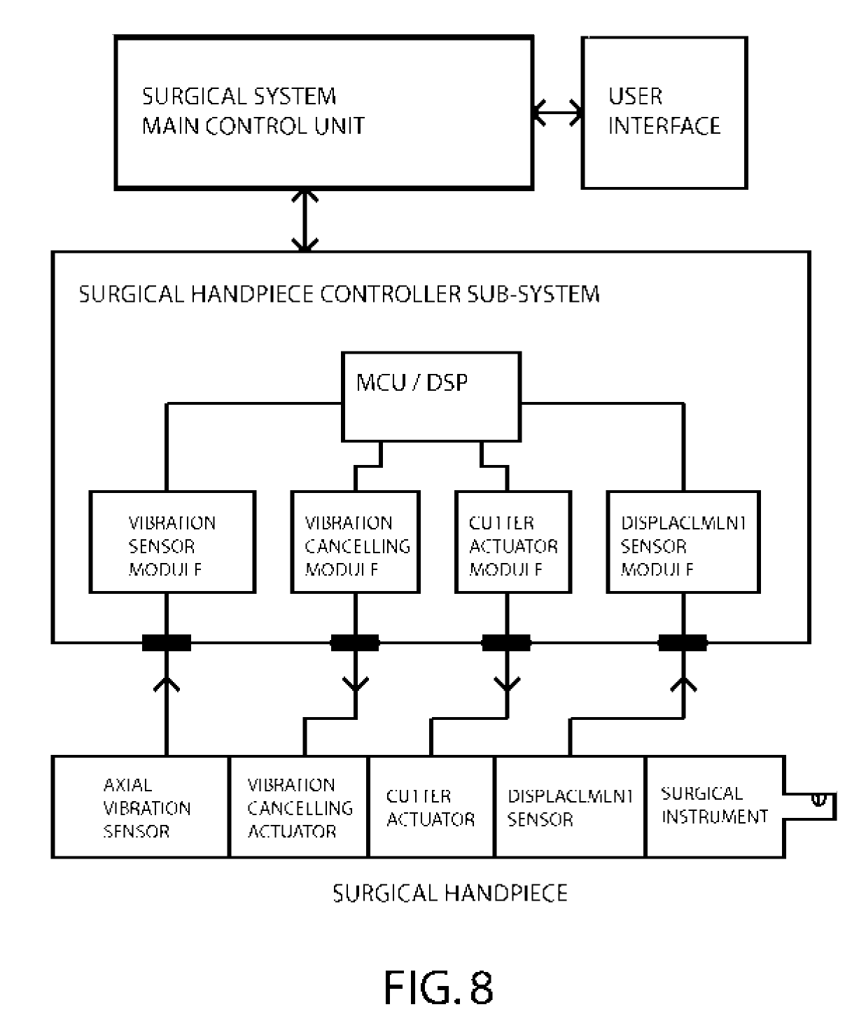

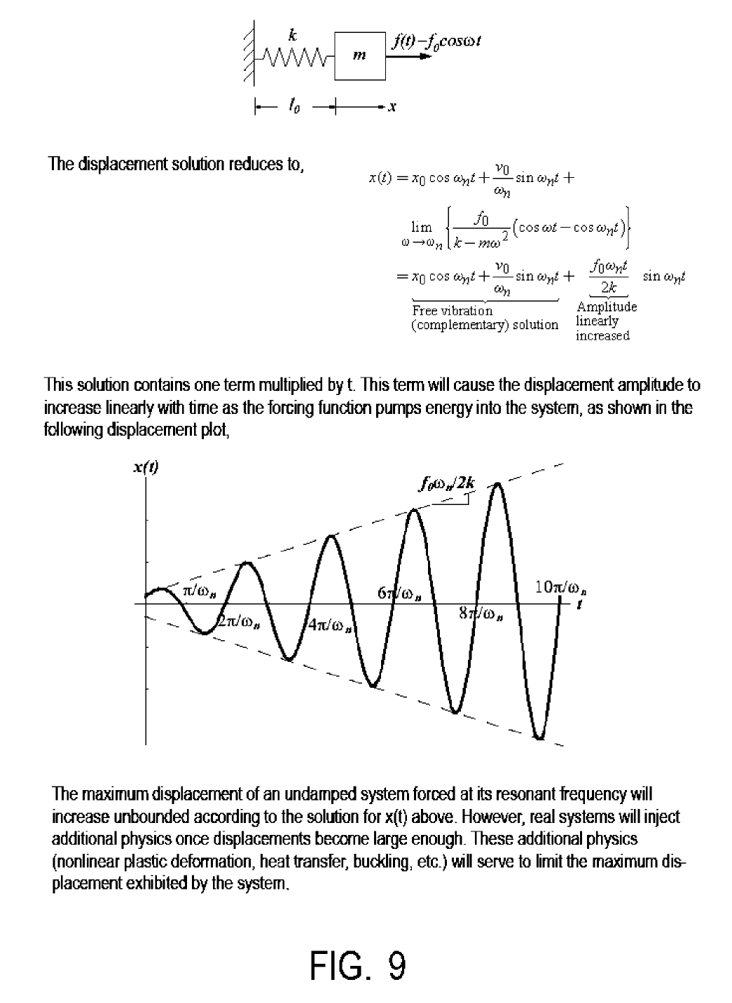

A surgical system incorporating a vitrectomy handpiece 10 of the present invention as shown in FIGS. 1 to 8 is composed of a vitrectomy console 70 including a user interface 71 with operator controls 72 and a display 73. A source of pressurized balanced salt solution 90 can be delivered into an eye 96 through an infusion tubing 94 placed across a solenoid 92 and into an irrigation incision 97 of an eye 96. A footpedal 74 is connected to console 70 through a cable 75 and a connector 76.

Console 70 can also provide to vitrectomy handpiece 10 a source of vacuum through a connector 77 and an aspiration tubing 19 inserted into an aspiration port 18, with vitrectomy handpiece 10 eventually inserted into eye 96 through a vitrectomy incision 98. A connector 78 provides electric communication between console 70 across electric conductor cables 20, 21, 22, 23 with actuators 30, 310, 408 and sensor elements 80, 410 inside a body 16 of handpiece 10. Referring now to FIGS. 1 and 2, handpiece 10 of the present invention is composed of a body 16 and a detachable head 17.

Detachable head 17 includes a hollow vitrectomy probe 12 having a proximal end 11 and a distal end 13. A vitrectomy sideport 14 is preferably located near vitrectomy probe 12 distal end 13. Aspiration port 18 is in fluid communication with sideport 14 through a tubing 42.

Aspiration port 18 can connect through aspiration tubing 19 and connector 77 with an aspiration source provided by vitrectomy console 70. The vitreous cutting mechanism of handpiece 10 of the present invention is activated by the action of piezoelectric electro-mechanic actuators. It is known fact that typical single element or stack based piezoelectric actuators provide high force but limited displacement.

The guillotine cutter of a vitrectomy handpiece will require a stroke above 700 microns to fully displace across a typical vitrectomy sideport. This stroke cannot be achieved using direct piezoelectric actuators in a typical configuration within the practical dimensions and weight of a standard vitrectomy handpiece. This invention is based on the use of conventional or leveraged piezoelectric actuators to activate a vitrectomy handpiece.

Direct actuators such as Cedrat PPA-20M Parallel Pre-Stressed actuator or amplified piezoelectric actuators such as Cedrat APA50XS can be used with advantage in this application (Cedrat Technologies, 15 Chemin de Malacher, ZIRST, 38246 Meylan Cedex, France, http://www.cedrat.com). Also, piezoelectric actuators based on telescopic architectures or disk translators, such as P-288 HVPZT provided by Physik Instrumente can be used. Each of these architectures has its characteristic static, quasi-static and dynamic properties and can be used in different embodiments of this invention.

The required stroke for a typical vitrectomy guillotine is above 700 microns. Piezoelectric actuators produce small strokes with high force. The present invention uses a piezoelectric actuator to produce harmonic excitation of a spring-mass system amplifying the stroke to operate a vitrectomy handpiece. Proper selection of spring characteristics, mass, and dampening allows operation of the vitrectomy guillotine at the desired stroke and frequency. In the preferred embodiment for the present invention handpiece body 16 contains a piezoelectric actuator 310 receiving cable 21 at connector 32. One end of piezoelectric actuator 310 is fixed to handpiece body 16, while the opposing free end of piezoelectric actuator 310 is coupled with a mass 306 through a spring 300. Mass 306 connects through a connector/coupling 306 with a guillotine 44 having a cutting border 15.

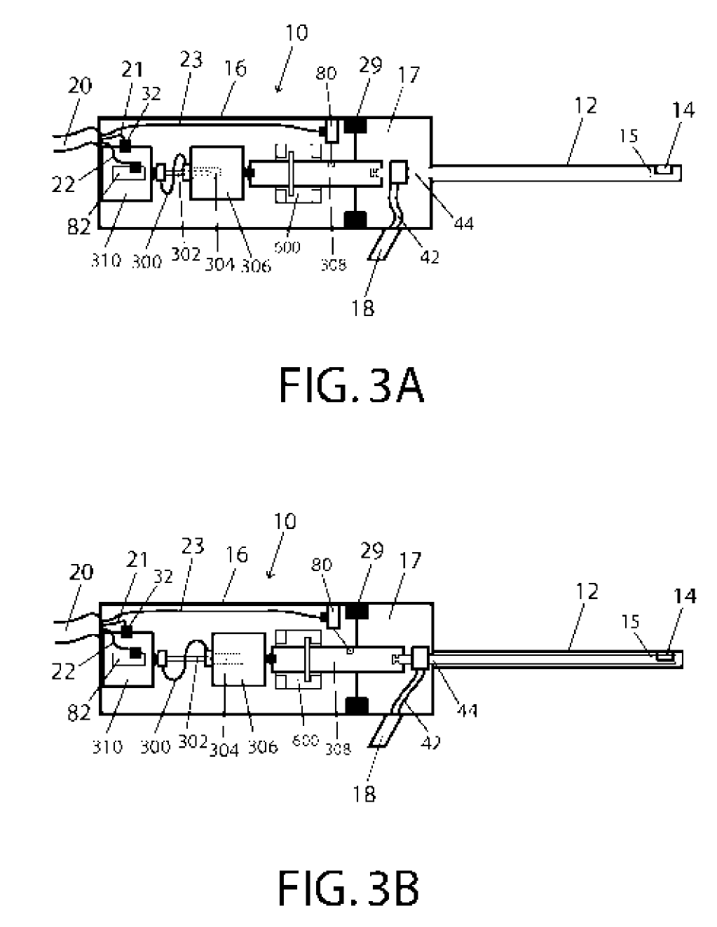

A stopper/damper mechanism 600 fixated to handpiece body 16 can be incorporated to regulate system dynamics at resonant frequency. An optional male guide 302 fits in a complementary female guide 304 within mass 304 to allow a single degree of freedom (DOF) of displacement of mass 304 in the axis of operation of piezoelectric actuator 310. As depicted in FIGS. 3 to 7, detachable head 17 includes hollow vitrectomy probe 12 with an internally disposed guillotine cutter 44 with a cutting border 15 sliding with a cutting action across the inner aspect of sideport 12. When not occluded by guillotine cutter 44, sideport 12 is in fluid communication with aspiration port 18 through an aspiration channel inside hollow vitrectomy needle 12, and fluid connector 42.

Aspiration port 18 can be connected to a vacuum source typically provided by vitrectomy console 70. Hollow vitrectomy needle 12, guillotine 44, aspiration port 18 and vacuum connector 42 are incorporated into handpiece head 17 that can be detachably connected to operate in conjunction with handpiece body 16. Head 17 is detachably connected using an attachment mechanism 19 preferably based on a bayonet or threaded coupling.

The position sensor element 80 can be constituted by one or more strain gauges, Eddy current sensors, capacitive position sensors, optical position sensors, LVDTs or any other position sensor elements suitable to detect in real time the axial position and displacement information of the oscillating spring-mass mechanism and of the driving piezoelectric actuator. Position sensor element 80 connects to console 70 sequentially through cables 23, 20 and connector 78.

Piezoelectric actuator 310 can incorporate a position sensor 82 preferably in the form of a strain gage to inform a controller system the displacement of the actuator independently of the displacement of the complete spring mass system. Position sensor 82 connects to console 70 sequentially through cables 22, 20 and connector 78.

During operation, an operator holds handpiece 10 by its body 16 and the hollow vitrectomy needle 12 can be inserted into an eye 96 through an incision 98. An aspiration source can be connected to port 18 in fluid communication with cutting port 14. Irrigation solution can be provided to the interior of eye 96 through an irrigation line 94 using an irrigation incision 97. Following an operator commands a suitable electrical signal is provided by vitrectomy console 70 through cables 20 and 21, the voltage typically ranging between −20 and +150 volts and following a sine-wave.

According to the piezoelectric effect, a varying voltage level will make the piezoelectric actuator 310 to axially expand and contract describing a sinusoidal path with a stroke proportional to the amplitude of the applied driving signal. For a typical direct piezoelectric actuator for use in this application, the maximum stroke can reach 20 microns. The axial displacement of actuator 310 is transmitted to the spring-mass system composed by spring 300, mass 306 and the mass added by coupling 306 and guillotine 44.

An optional damper and stopper mechanism is conformed between the body of coupling 306 and handpiece body 16. This miniature damper is preferably designed to operate in viscous under-damped modality. Shear forces and the under-dampening effect of the damper/stopper mechanism 600 are considered for tuning the system for operation.

FIG. 11 depicts the formulas and dynamics that apply to the spring-mass mechanism of operation of the present invention. It is desirable that the spring-mass system is un-damped or under-damped to operate continuously at harmonic frequency. At design time, stiffness of spring 300 and the value of the total mass of the spring-mass system together with any present damping forces are determined to operate in harmonic excitation at a selected frequency of operation, with a desired stroke.

As a mode of example only, by selecting a spring with a stiffness of 1 N/mm and a total mass of 10 grams, the system will have its first resonant frequency at 50.3 Hertz, allowing a guillotine cutter system to operate at approximately 3.000 cuts per minute. The PPA-20M actuator has a blocked-free resonating frequency of 21.250 Hertz. For this reason, to operate the system at 50.3 Hertz in the first resonant frequency, the actuator is driven in non-resonant mode to provide 20 microns of sinusoidal displacement at 50.3 hertz. In this way, the spring-mass system composed by spring 300, mass 306, and the masses of coupling 306 and of guillotine 44 are subjected to harmonic excitation, oscillating at amplitudes that are approximately 40 times bigger than the amplitude of oscillation of the excitation actuator 310 to achieve an axial stroke of guillotine 44 of 800 microns.

An optional displacement sensor 80 can be used to continuously monitor operation of the handpiece by the surgical handpiece controller system to determine proper oscillation of guillotine 44. Shifts in resonant frequency of the spring-mass system are corrected at controller level to maintain the stroke at a constant level during operation. Also, changes in the stroke of guillotine 44 are adjusted by modifying the driving signal provided to the piezoelectric actuator. Considering a stroke amplification of 40 times to obtain 800 microns guillotine stroke from a piezoelectric actuator providing 20 microns stroke, a proper combination of spring stiffness and total mass for the spring-mass system is selected at design time to operate at a desired frequency. In a simple mode of operation, the system is adjusted to have the cutting border 15 midway across sideport 14 in resting position. Once activated, the resonant system oscillates around this center point to the fully open and fully closed position to perform the vitreous aspiration and cutting action. This modality provides a sideport 14 open-to-closed ratio of 1/1 (or 50% duty cycle) and leaves sideport 14 half closed when not oscillating.

To increase the open-to-closed ratio and also to provide a sideport 14 that is fully open when guillotine 15 is not oscillating, an offset can be applied to the cutting border 15 in resting position. This mode of operation requires an increase in stroke for proper operation up to 100%, but provides a fully open sideport 14 when guillotine 44 is not oscillating, and can also increase sideport 14 open-to-close ratio 2/1 (or 66% duty cycle) or above. A piezoelectric actuator controller system can keep track of proper operation of the actuator-spring-mass system by monitoring mass position sensor 80 and/or piezoelectric actuator position sensor 82.

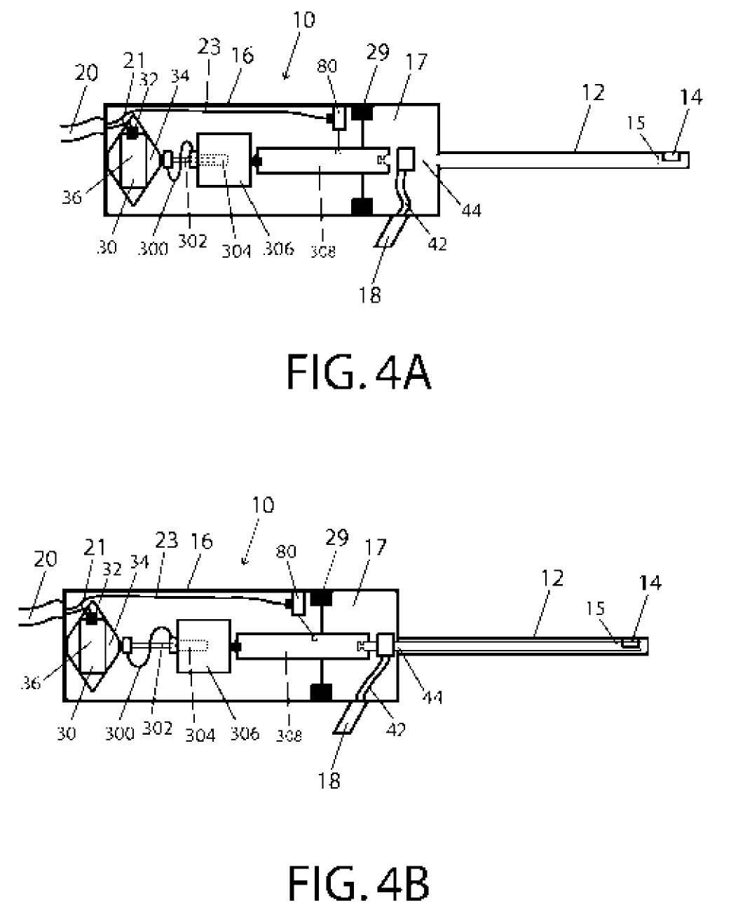

As depicted in FIGS. 4A and 4B, an amplified piezoelectric actuator 30 can be used instead of a direct piezoelectric actuator. In this configuration, the leveraged piezoelectric actuator has a piezoelectric element 36 perpendicularly disposed inside a frame 34. Sinusoidal activation of the piezoelectric element 36 produces a sinusoidal oscillation of the amplified actuator with increased stroke. As a mode of example, using Cedrat's APA50XS amplified piezoelectric actuator can produce a stroke up to 80 microns. By using this kind of actuator, the stiffness of spring 300 and the magnitude of the total mass of the spring-mass system, including mass 306 can be recalculated with improved performance.

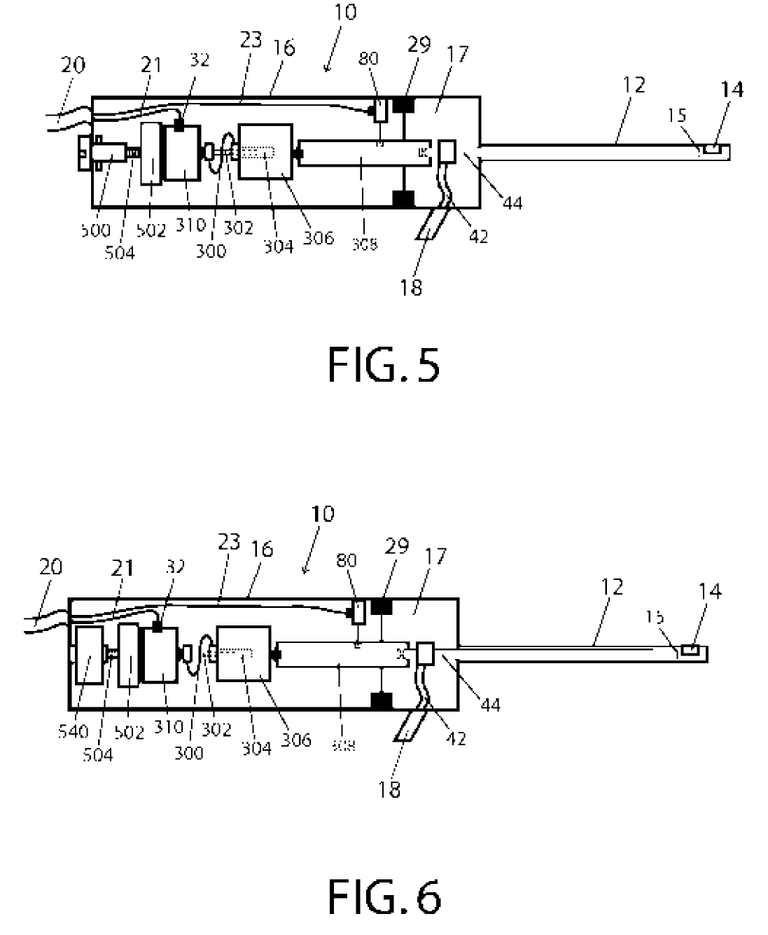

FIG. 5 depicts an alternative embodiment incorporating an adjustment knob 500 with a female thread receiving a male thread 504 extending from support 502 holding piezoelectric actuator 301. This configuration allows an operator to adjust the axial position of actuator 310, spring-mass, coupling 306 and guillotine 44. In this way the relative position of guillotine 15 with respect to sideport 14 can be regulated, modifying the maximally open dimensions of sideport 14 to accommodate to different surgical conditions.

FIG. 6 depicts another alternative embodiment replacing the manual adjustment knob 500 depicted in FIG. 5 with a miniature linear actuator 540. Linear actuator 540 can axially displace 502 holding piezoelectric actuator 301. This configuration allows adjustment of the axial position of actuator 310, spring-mass, coupling 306 and guillotine 44 under controller command. In this way the relative position of guillotine 15 with respect to sideport 14 can be regulated, modifying the maximally open dimensions of sideport 14 to accommodate to different surgical conditions. Linear actuators suitable for this application are miniature actuators such as Smoovy Series 06A S2, from MicroMo Electronics, 14881 Evergreen Ave. Clearwater, Fla. 33762-3008, USA. Console controlled operation of linear actuator 540 can also allow proportional operation of surgical handpiece 10.

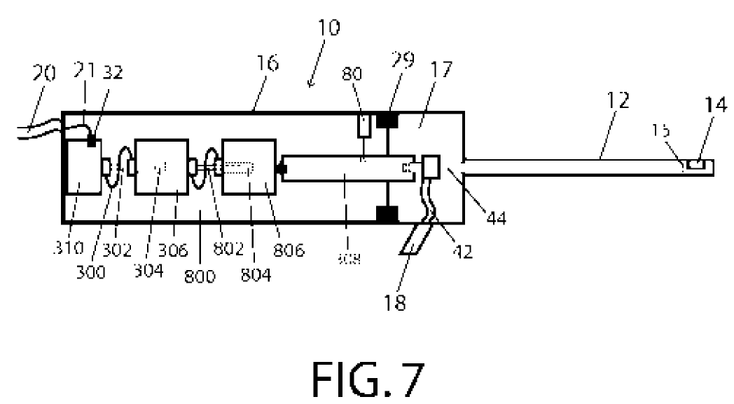

FIG. 7 depicts another embodiment with a spring mass-system incorporating a second spring 800 and mass 806, with guides 802 and 804. In this configuration both masses 306 and 806 oscillate along the same axis in mirror fashion. This structure and modality of operation is aimed to reduce handpiece 10 unwanted axial vibration during operation.

Thus the reader will understand that the surgical system of the invention improves over the prior art by providing a surgical handpiece that incorporates a surgical handpiece powering method based on piezoelectric harmonic excitation of a spring-mass system. The introduction of a piezoelectric actuator driven spring-mass system for the operation of the handpiece allows high speed of operation. Complementary offset adjusting mechanism allows regulation of sideport functional dimensions. While the above description provides many specificities these should not be construed as limitations on the scope of the invention, but rather as exemplifications of preferred embodiments.

For example, the illustrated piezoelectric actuator can be replaced by other architectures of piezoelectric actuators according to stroke, force and dynamic requirements for a particular system without departing from the scope of the present invention.

Activation of the handpiece can be made using a footpedal, sensors in the handpiece or other suitable surgical instrument operator activation method.

The controller of the handpiece can be located within the same handpiece using microelectronic circuits instead of a console located controller.

The probe head can be detachable or permanently assembled to the handpiece body. Accordingly, the scope of the present invention should be determined not by the embodiments illustrated but by the appended claims and their legal equivalents.

While only certain preferred features of the invention have been illustrated and described, many modifications, changes and substitutions will occur to those skilled in the art. It is, therefore, to be understood that the appended claims are intended to cover all such modifications and changes as fall within the true spirit of the invention.

I claim:

1. A spring-mass based piezoelectric surgical system, comprising:
a surgical instrument actuated by oscillatory motion; wherein
said oscillatory motion is produced by a spring-mass system oscillating proximate a harmonic frequency of the spring-mass system and at least one piezoelectric actuator coupled thereto, said at least one piezoelectric actuator actuated proximate said harmonic excitation frequency of said spring-mass system.

2. The system of claim 1, further comprising:
said spring-mass system; and
said at least one piezoelectric actuator coupled thereto.

3. The system of claim 2, further comprising:
a surgical handpiece coupled to said surgical instrument, comprising said spring-mass system and said at least one piezoelectric actuator.

4. The surgical system of claim 3, further comprising:
a surgical handpiece controller system coupled to and controlling said surgical handpiece and said surgical instrument.

5. The system of claim 1, said surgical instrument comprising a guillotine-based vitrectomy probe.

6. The system of claim 1, said piezoelectric actuator comprising a parallel pre-stressed piezoelectric actuator.

7. The system of claim 1, said piezoelectric actuator comprising an amplified piezoelectric actuator.

8. The system of claim 3, said surgical handpiece further comprising sensor means to detect a linear displacement produced by said piezoelectric actuator.

9. The system of claim 3, said surgical handpiece further comprising sensor means to detect the linear displacement produced by the spring-mass system.

10. The system of claim 3, said surgical handpiece further comprising axial vibration canceling means for canceling axial vibrations.

11. The system of claim 1, wherein said system is operable in closed-loop servo control modality.

12. The system of claim 5, said guillotine based vitrectomy probe comprising open sideport area adjusting means for adjusting an open sideport area of said guillotine based vitrectomy probe.

13. The system of claim 12, said open sideport area adjusting means further comprising a linear actuator.

14. A method for powering a surgical instrument comprising using a piezoelectric actuator driving a spring-mass system at a harmonic excitation frequency of said spring-mass system for powering said surgical instrument.

15. The method of claim 14, said surgical instrument comprising a vitrectomy probe.
